SEQUENCE LISTING (5 sequences)

SEQ ID NO: 1 - protein, 39 aa, Artificial Sequence
  Description of Artificial Sequence Synthetic polypeptide
SYSMEHFRWGKPVGKKRRPVKVYPNGAEDESAEAFPLEF

SEQ ID NO: 2 - protein, 24 aa, Artificial Sequence
  Description of Artificial Sequence Synthetic peptide
SYSMEHFRWGKPVGKKRRPVKVYP

SEQ ID NO: 3 - protein, 16 aa, Artificial Sequence
  Description of Artificial Sequence Synthetic peptide
SYSMEHFRWGKPVGKK

SEQ ID NO: 4 - protein, 17 aa, Artificial Sequence
  Description of Artificial Sequence Synthetic peptide
SYSMEHFRWGKPVGKKR

SEQ ID NO: 5 - protein, 39 aa, Artificial Sequence
  Description of Artificial Sequence Synthetic polypeptide
SYSMEHFRWGKPVGKKRRPVKVYPDGAEDQLAEAFPLEF

(19) United States
(12) Patent Application Publication
(10) Pub. No.: US 2018/0169191 A1
(43) Pub. Date: Jun. 21, 2018

(54) ADRENOCORTICOTROPIC HORMONE-BASED PHARMACEUTICAL FORMULATIONS AND METHODS FOR FABRICATING AND USING THEREOF

(71) Applicant: ETON Pharmaceuticals, Inc., Deer Park, IL (US)

(72) Inventors: Dennis Elias Saadeh, Irvine, CA (US); Mark L. Baum, San Diego, CA (US)

(21) Appl. No.: 15/621,653

(22) Filed: Jun. 13, 2017

Related U.S. Application Data
(60) Provisional application No. 62/436,082, filed on Dec. 19, 2016.

(51) Int. Cl. A61K 38/35; A61K 9/00; A61K 47/38; A61K 47/26; A61K 47/18; A61K 47/34

(52) U.S. Cl. CPC A61K 38/35; A61K 9/0019; A61K 47/34; A61K 47/26; A61K 47/183; A61K 47/38

(57) ABSTRACT

Pharmaceutical compositions for treating, mitigating or preventing multiple sclerosis, autoimmune diseases and associated conditions are described herein. Methods for fabricating the compositions and using them are also described.

### CROSS-REFERENCE TO RELATED APPLICATION(S)

This application claims priority under 35 U.S.C. § 119(e) to U.S. Provisional Application No. 62/436,082, filed on Dec. 19, 2016, the entire content of which is hereby incorporated by reference.

### SEQUENCE LISTING

The instant application contains a Sequence Listing which has been submitted electronically in ASCII format and is hereby incorporated by reference in its entirety. The ASCII copy, created on Jun. 13, 2017, is named 20243-101553_SL.txt and is 2,239 bytes in size.

### FIELD OF THE INVENTION

The present invention relates generally to the field of treating multiple sclerosis, autoimmune diseases and associated conditions, and more specifically to corticotropin-based compositions, to methods of using the compositions to treat, mitigate or prevent such diseases, and to methods of preparing such compositions.

### BACKGROUND

A significant portion of the population worldwide suffers from multiple sclerosis, autoimmune diseases (e.g., lupus), and associated conditions such as infantile spasms, Addison's disease, Nelson's, Cushing's and West syndromes, etc.

Typical current treatments of such diseases include the use of animal-derived corticotropin. One of commonly used composition utilizing corticotropin is known under the trade name ACTHAR®, available from Mallincrodt Pharmaceuticals (St. Louis, Mo.), and under other trade names. While naturally occurring, animal-derived corticotropin may have certain beneficial properties, the existing corticotropin-based treatments, however, are of limited effectiveness in many patients, due in part to frequent and quite severe side effects that include difficulty with breathing, dizziness, tightness in the chest, slow wound healing, increased sweating, headache, spinning sensation, and more. In some cases, side effects may be of such severity that the treatment has to be discontinued.

Accordingly, there exists a need for improved methods and compositions for treatment, mitigation and/or prevention of autoimmune diseases and associated conditions. This patent specification discloses such pharmaceutical compositions that would achieve positive patient outcomes while being free of drawbacks and deficiencies of existing formulations, and methods of fabricating and administering the same.

### SUMMARY

According to one embodiment of the invention, a pharmaceutical composition is provided. The composition comprises, consists essentially of, or consists of corticotropin of a non-animal derivation, wherein the composition is free of gelatin and free of preservatives.

According to another embodiment of the invention, the corticotropin used in the composition described herein comprises a quantity of one or several recombinant polypeptide(s) having the amino acid sequence(s) described hereinbelow; and may further optionally contain a quantity of a naturally occurring polypeptide.

According to other embodiments of the invention, there are provided methods for treating, preventing or alleviating a disease, condition, syndrome, symptom, pathology, or malady using such corticotropin-based compositions, the diseases including multiple sclerosis, autoimmune diseases (e.g., lupus), rheumatoid disorders, infantile spasms, Addison's disease, Nelson's, Cushing's and West syndromes.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary only and are not restrictive of the invention as claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees, depending on the context. Whenever it appears herein, a numerical range such as “1 to 20” or “1-20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of a disease or pathology.

The terms “adrenocorticotropic hormone” (sometimes abbreviated hereinafter as “ACTH”) and corticotropin are used hereinafter interchangeably and refer to a polypeptide tropic hormone produced by the anterior pituitary gland of a mammal. The structure and some basic properties of ACTH are discussed in more detail below. It is further provided that for the purposes of the present disclosure, a fully synthetic version of corticotropin (also discussed in more detail below) is considered within the scope of meaning attributed to ACTH and corticotropin.

The term “polypeptide” is defined as a linear organic polymer consisting of between about 10 and about 50 amino acid residues bonded together in a chain via amide bonds.

The term “recombinant polypeptide” for the purposes of this application means a polypeptide obtained synthetically (i.e., artificially, both chemically and otherwise) that is identical in every respect to the corresponding polypeptide of the same amino acid sequence that occurs naturally in living organisms.

The term “autoimmune disease” refers to a pathological physiological state or condition in a human body that arises from abnormal acquired immune system's reactions of the body to substances and/or tissues that are normally present in the body.

The term “rheumatic disorder” refers to a variety of pathological physiological states or conditions in a human body that cause chronic pain affecting the joints and/or connective tissue. As used herein, this term is inclusive of both the disorders primarily caused by autoimmunity (e.g., rheumatoid arthritis, juvenile arthritis, gout, etc.) and the disorders of other origins (e.g., bursitis, osteoarthritis, spondylitis, etc.).

The terms “infantile spasm” and “West syndrome” refer interchangeably to a specific type of seizure seen in an epilepsy syndrome of infancy and is characterized by developmental regression and by hypsarrhythmia (chaotic brain waves), which is a specific pattern on electroencephalography chart.

The term “Addison's disease” refers to an endocrine disorder in which the adrenal glands of the sufferer do not produce enough steroid hormones.

The term “Cushing's syndrome” refers to a condition characterized by increased secretion of adrenocorticotropic hormone resulting in multiple negative effects on the health of the patient, e.g., weight gain, hypertension, impaired immunological function, etc.

The term “Nelson's syndrome” refers to a disorder resulting from an adrenalectomy performed for Cushing's disease, wherein the patient develops macroadenomas that secrete adrenocorticotropic hormone resulting in various severe health problems.

The term “lupus” refers to a chronic inflammatory disease that occurs when a person's immune system attacks its own tissues and organs with severe and potentially deadly effects on many body systems, including blood cells, brain, heart, joints, skin, kidneys, and lungs.

The term “therapeutically effective amount” is defined as the amount of a compound or pharmaceutical composition that will elicit the biological or medical response of a tissue, system, animal or human, that is being sought by the researcher, medical doctor or other clinician.

The term “potency” is defined in accordance with the U.S. Pharmacopeia and refers to the quantity of an active pharmaceutical component in the composition. In accordance with the requirements of the U.S. Pharmacopeia, the potency that is required of the corticotropin compositions described below is not less than 80.0 percent and not more than 125.0 percent of the potency stated on the label in USP corticotropin units.

The term “pharmaceutically acceptable” when used to describe a carrier, whether diluent or excipient, is defined as being compatible with the other ingredients of the formulation and not deleterious to the recipient thereof.

The terms “administration of a composition” or “administering a composition” are defined to include the act of providing a compound or pharmaceutical composition of the invention to the subject in need of treatment.

### B. Embodiments of the Invention

According to embodiments of the present invention, pharmaceutical compositions are provided for treating, mitigating or preventing various diseases, syndromes and maladies described below. The compositions include corticotropin of a non-animal derivation, and are free of both gelatin and of any preservatives, e.g., are free of such a preservative as phenol. While some of ordinary skill in the art may consider cysteine or its related amino acids (e.g., methionine) as being among preservatives used in some applications, nevertheless, for the purposes of the instant disclosure, cysteine and related amino acids are defined as not being a preservative. The compositions of the present invention, therefore, may contain cysteine or related amino acids such as methionine, or, alternatively, may be cysteine-free and free of amino acids related to cysteine (such as methionine), as desired.

Corticotropin exists in two versions. The first version of ACTH is a naturally occurring ACTH that is a product of proteolytic cleavage (by the prohormone convertase) of the prohormone, proopiomelanocortin, which is secreted from corticotropes in the anterior lobe of the pituitary gland. Thus, ACTH is formed as a polypeptide tropic hormone having a 39-amino acid sequence and an average molecular weight of about 4,540 Daltons.

The second version of ACTH employed in the compositions described herein is a fully synthetic, recombinant polypeptide having the same 39-amino acid sequence as the natural version. There are several known ways of synthesizing the recombinant polypeptide and the recombinant polypeptide obtained by using any of these methods may be utilized. In addition to the full synthetic analog of natural ACTH (both having the same 39-amino acid sequence), polypeptides having the first 24, first 17, and first 16 amino acid residues of the 39-amino acid sequence may be obtained and used as described below.

Therefore, according to embodiments of the invention, corticotropin in the pharmaceutical compositions comprises a quantity of a recombinant polypeptide and some quantity of a naturally occurring polypeptide. In other embodiments, corticotropin in the pharmaceutical compositions comprises a quantity of a recombinant polypeptide only, and is, therefore, completely free of the naturally occurring polypeptide or contains not more than a negligible trace amount of the naturally occurring polypeptide.

The recombinant polypeptide in the composition is a polypeptide that includes the 39-amino acid sequence as set forth in SEQ ID NO: 1, as shown below:

(SEQ ID NO: 1)
SYSMEHFRWGKPVGKKRRPVKVYPNGAEDESAEAFPLEF

The recombinant 39-amino acid sequence as set forth in SEQ ID NO: 1, shown above, is the same sequence that is found in the naturally occurring ACTH.

If desired, any of the 24-amino acid polypeptide as set forth in SEQ ID NO: 2, the 16-amino acid polypeptide as set forth in SEQ ID NO: 3, the 17-amino acid polypeptide as set forth in SEQ ID NO: 4, as well as another 39-amino acid as set forth in SEQ ID NO: 5, discussed in more detail below, and any combination thereof, may be present in the composition in addition to, or, if desired, instead of, the 39-amino acid polypeptide of SEQ ID NO: 1. SEQ ID NO: 2, SEQ ID NO: 3, and SEQ ID NO: 4 are as follows:

(SEQ ID NO: 2)
SYSMEHFRWGKPVGKKRRPVKVYP
(SEQ ID NO: 3)
SYSMEHFRWGKPVGKK
(SEQ ID NO: 4)
SYSMEHFRWGKPVGKKR

The amino acid residues in each of SEQ ID NO: 2, SEQ ID NO: 3, and SEQ ID NO: 4, are the first 24, the first 16, and the first 17 amino acid residues, respectively, of the 39-amino acid sequence of naturally occurring ACTH and in the recombinant polypeptide as set forth in SEQ ID NO: 1.

As mentioned above, according to yet other embodiments, another 39-amino acid recombinant polypeptide can be used in the composition, as shown below:

(SEQ ID NO: 5)
SYSMEHFRWGKPVGKKRRPVKVYPDGAEDQLAEAFPLEF

As one may observe, SEQ ID NO: 5 differs from SEQ ID NO: 1 by three amino acid residues (at positions 25, 30, and 31), i.e., by having aspartic acid (D) at position 25, glutamine (Q) at position 30, and leucine (L) at position 31, while the polypeptide of SEQ ID NO: 1 has asparagine at position 25 (N), glutamic acid (E) at position 30, and serine (S) at position 31. As such, SEQ ID NO: 5 may be used in compositions that are the subject of the present disclosure, alone or in any combination with any or all of SEQ ID NO: 1, SEQ ID NO: 2, SEQ ID NO: 3, and SEQ ID NO: 4.

The quantity of the recombinant polypeptide in the composition may be between about 0.001 and about 0.010 mass %, such as between about 0.002 and about 0.008 mass %, for example, about 0.006 mass %. Of the entire recombinant polypeptide, the 39-amino acid polypeptide of SEQ ID NO: 1, if used, constitutes as least about 50.0 mass %.

As mentioned above, the pharmaceutical compositions described herein are preservative free but may further optionally contain various inactive components. Some non-limiting examples of inactive components that may be so used include at least one inert water-soluble thickening agent such as dextrose, mannitol, or carboxymethyl cellulose. Some non-limiting example(s) of additional water-soluble thickening agent(s) that may be used instead of, or in addition to, dextrose, mannitol and/or carboxymethyl cellulose are cysteine, methyl cellulose, hydroxyethyl cellulose, hydroxypropyl cellulose, non-cross-linked or partially cross-linked polyacrylates, polyoxyethylene sorbitan monolaurates, polyoxyethylene sorbitan monopalmitates, polyoxyethylene sorbitan monostearates, and polyoxyethylene sorbitan monooleates.

The concentration of the water-soluble thickening agent(s) described above, if used in the compositions, may be between about 0.5 mass % and 2.0 about mass % of the total mass of the composition, such as between about 1.0 mass % and about 1.5 mass %, for example, about 1.25 mass %.

According to further non-limiting embodiments, the preservative-free pharmaceutical compositions described herein may further optionally contain additional inactive component(s) that may be used in combination with, or instead of, the inactive component(s) described above. Non-limiting example(s) of such additional inactive component(s) include at least one non-ionic poly(oxyethlene-co-oxypropylene) block copolymer having the following general structure:

HO—(CH₂—CH₂—O)ₓ—(C₃H₆—O)_{y}—(CH₂—CH₂—O)ₓ—H,

wherein in some further non-limiting embodiments, x is an integer that can have the value of at least 8 and y is an integer that can have the value of at least 38.

One non-limiting example of an even more specific non-ionic poly(oxyethlene-co-oxypropylene) block copolymer that can be used is the product known under the trade name Poloxamer 407® (poly(ethylene glycol)-block-poly(propylene glycol)-block-poly(ethylene glycol)) available from Sigma-Aldrich Corp. of St. Louis, Mo. (the trade name is owned by BASF Corp.), with the molecular weight of the polyoxypropylene portion of about 4,000 Daltons, about a 70% polyoxyethylene content, the overall molecular weight of between about 9,840 Daltons and about 14,600 Daltons and having the following chemical structure

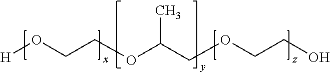

Other similar products of the Poloxamer® family are useful for forming the thermoreversible gel of the compositions of the present invention are, for example, the products of the Pluronic® family, Kolliphor® family (the trade names are also owned by BASF) or Synperonics® family (Croda International plc). Any polymer of Poloxamer®, Pluronic®, Kolliphor® or Synperonics® family that is used may contain any portion that is cross-linked.

The concentration of the non-ionic poly(oxyethlene-co-oxypropylene) block copolymer(s) described above, if used in the compositions, may be between about 0.1 mass % and about 2.0 mass % of the total mass of the composition, for example, about 1.0 mass %.

According to further embodiments, methods for fabricating the above-described pharmaceutical articles are provided. A one-batch formulation method may be used, where the components of the pharmaceutical formulation are combined in single container; the components may be added to the container simultaneously or consecutively.

The resulting product may then be adapted for administration by a suitable route (e.g., by intramuscular or subcutaneous injection), and may then be prescribed and given to a patient for treating, mitigating or preventing multiple sclerosis and autoimmune diseases, as well as associated syndromes, symptoms, pathologies, maladies, or conditions, such as comprise infantile spasms, Addison's disease, Nelson's, Cushing's and West syndromes.

It will be understood by those having ordinary skill in the art that the specific dose levels and frequency of administration for any particular patient may be varied and will depend upon a variety of factors including the activity of the specific compound employed, the metabolic stability and length of action of that compound, the age, body weight, general health, gender, diet, and the severity of the particular disease, syndrome, symptom, pathology, malady, or condition being treated.

It is also noteworthy that if an amino acid is used in compositions, the choice and concentration of the amino acid may be consequential. For example, in some experiments cysteine was used at a concentration of about lmg/ml. Such compositions lacked sufficient potency. On the other hand, using methionine instead of, or in addition to, cysteine, combined with increased concentration of the amino acid(s) up to about mg/ml can improved the potency substantially and to bring it to desirable level. Using mannitol has also proved to be beneficial for increasing the potency. Some comparative data regarding the potency of some compositions is provided below in the “Examples” portion of the application.

In additional embodiments, the above described pharmaceutical formulations may be incorporated within microparticles. The microparticles may be substantially spherical particles (shells) fabricated of a water soluble biodegradable polymer defining a space therein, which space is to be filled with the pharmaceutical formulation. Thus, the microparticles represent the structures where the water soluble biodegradable polymer envelops the formulation securely ensconcing the latter and not allowing the formulation to prematurely escape or to leak out.

The formulation-filled microparticles can be manufactured according to methods and techniques known to those having ordinary skill in the art. The size of microparticles may be typically less than about 100 μm in diameter, and the exemplary water soluble polymer to be used to manufacture the shells may be, without limitations, any of poly(lactic acid-co-glycolic acid), poly(lactic acid), poly(glycolic acid), poly(caprolactone), poly(hydroxybutyrate) and blends thereof. In one typical example, poly(lactic acid-co-glycolic acid) can be used to form the shells, with the 50:50 (mass) ratio between the units derived of lactic and glycolic acids. Other acceptable ratios between the lactic and glycolic acid portions may be 65:35, 75:25 and 85:15. Those having ordinary skill in the art and using poly(lactic acid-co-glycolic acid) may select a different ratio, if desired. The inherent viscosities (i.e., the ratio of the natural logarithm of the relative viscosity to the mass concentration of the polymer) of the polymer solutions used to form the shells may be between about 0.15 dL/g and about 1.20 dL/g, such as between about 0.15 dL/g and 0.25 dL/g, or of the following ranges: 0.26-0.54, 0.55-0.75, 0.62-0.65, 0.65-0.85, 0.76-0.94 and 0.95-1.20 dL/g.

When the above described microparticles have been fabricated, they can then be administered to a patient in need of the medication by conventional methods described herein, such as by injection.

In additional embodiments, pharmaceutical kits are provided. The kits include sealed containers approved for the storage of pharmaceutical compositions, and the above-described pharmaceutical composition. An instruction for the use of the composition and the information about the composition are to be included in the kit.

The following examples are provided to further elucidate the advantages and features of the present invention, but are not intended to limit the scope of the invention. The examples are for the illustrative purposes only. USP pharmaceutical grade products were used in preparing the formulations described below.

### Example 1. Preparing a Pharmaceutical Composition No. 1

A pharmaceutical composition can be prepared as described below. The following products can be used in the amounts and concentrations specified:
(a) about 0.0627 g of powdered adrenocorticotropic hormone ACTH;
(b) about 0.2 g of powdered cysteine;
(c) about 5.0 g of powdered mannitol;
(d) about 50.0 mL of a 2.5% aqueous solution of sodium carboxymethyl cellulose;
(e) a quantity of a 10% aqueous solution of hydrochloric acid for adjusting pH; and
(f) about 100.0 mL of sterile water suitable for injections.

To prepare the composition, the stated quantities of cysterine and mannitol may be mixed with about 30% of water (about 30 mL) followed by adding ACTH to the solution which can then be filtered into a sterile 100 mL vial through a 0.22 micron Teflon filter. Finally, the stated amount of sodium carboxymethyl cellulose may be added to the vial followed by agitation to achieve dispersion. Alternatively, the dispersion may be achieved by using the method of “transferring from syringe to syringe” known to those having ordinary skill in the art.

The product so obtained can then be checked for the level of pH, and if this level is outside the range of about 4.5 to about 7.0, it can be adjusted by adding, dropwise, a quantity of hydrochloric acid that is necessary to achieve such an adjustment. The product can then be transferred into 1 mL sterile amber serum vials with about 0.2 mL overfill.

### Example 2. Preparing a Pharmaceutical Composition No. 2

A pharmaceutical composition was prepared as described in Example 1 using the same components in the same quantities and employing the same compounding procedure, except that cysteine can be replaced by about 0.298 g of powdered methionine.

### Example 3. Preparing a Pharmaceutical Composition No. 3

A pharmaceutical composition was prepared as described below. The following products were used in the amounts and concentrations specified:
(a) about 0.0627 g of powdered adrenocorticotropic hormone ACTH;
(b) about 0.298 g of powdered methionine;
(c) about 5.0 g of powdered mannitol;
(d) about 0.3 g of POLOXAMER®;
(e) about 50.0 mL of a 2.5% aqueous solution of sodium carboxymethyl cellulose;
(f) a quantity of a 10% aqueous solution of hydrochloric acid for adjusting pH; and
(g) about 100.0 mL of sterile water suitable for injections.

To prepare the composition, the stated quantities of methionine and mannitol were mixed with about 30% of water (about 30 mL) followed by adding ACTH to the solution which was then filtered into a sterile 100 mL vial through a 0.22 micron Teflon filter. Finally, the stated amount of sodium carboxymethyl cellulose was added to the vial followed by agitation to achieve dispersion. Alternatively, the dispersion may be achieved by using the method of “transferring from syringe to syringe” known to those having ordinary skill in the art. The product can then be transferred into 1 mL sterile amber serum vials with about 0.2 mL overfill.

### Example 4. Comparative Potency Data of Some Pharmaceutical Compositions

Several compositions were prepared according to the methods illustrated in Examples 1-3. Table 1 provides a comparison of their respective potencies. The terms “recombinant porcine” and “recombinant human” refer to synthetic ACTH having the same amino acid sequence that is occurring naturally in pigs and humans, respectively. As can be seen from the data presented in Table 1, using methionine provides potency results that are considered unexpected based on the review of the potency data shown by cysteine-containing compositions.

TABLE 1
Select Compositions and Their Potencies

| ACTH | Amino acid | Mannitol | pH | Potency |
|---|---|---|---|---|
| Recombinant | Cysteine, | None | 4.5-6.5 | Failed¹ after 30 days |
| porcine | 1 mg/mL | | | |
| Recombinant | Cysteine, | Yes, | 4.0-6.0 | Failed after 10 days² |
| porcine | 2 mg/mL | 50 mg/mL | | |
| Recombinant | Methionine, | Yes, | 4.0-6.0 | Failed after 10 days² |
| porcine | 2 mg/mL | 50 mg/mL | | |
| Recombinant | Cysteine, | Yes, | 4.5-6.5 | Failed after 60 days³ |
| human | 2 mg/mL | 50 mg/mL | | |
| Recombinant | Methionine, | Yes, | 4.5-6.5 | Passed⁴, stable after |
| human | 2 mg/mL | 50 mg/mL | | 180 days |
| Recombinant | Cysteine, | Yes, | 4.5-6.6 | Failed after 30 days |
| porcine | 2 mg/mL | 50 mg/mL | | |
| Recombinant | Methionine, | Yes, | 4.0-6.0 | Passed, stable after |
| porcine | 3 mg/mL | 50 mg/mL | | 180 days |

¹“Failed” refers to the potency after the testing being without the 80-125% range required by the U.S. Pharmacopeia
²Tested both at accelerated (room temperature) and refrigerated (real time) conditions; the failure applies to both conditions
³Followed by even more substantial failure by day 90 (when only about 16.9% of the label amount remaining intact)
⁴“Passed” refers to the potency after the testing being within the 80-125% range required by the U.S. Pharmacopeia

Although the invention has been described with reference to the above described embodiments, it will be understood that modifications and variations are encompassed within the spirit and scope of the invention. Accordingly, the invention is limited only by the following claims.

What is claimed is:

1. A pharmaceutical composition, comprising a quantity corticotropin of a non-animal derivation, wherein the composition is free of gelatin and free of preservatives.

2. The pharmaceutical composition of claim 1, wherein the corticotropin comprises a quantity of a recombinant polypeptide and optionally a quantity of a naturally occurring polypeptide.

3. The pharmaceutical composition of claim 2, wherein the corticotropin is free of the naturally occurring polypeptide.

4. The pharmaceutical composition of claim 3, wherein the recombinant polypeptide is the amino acid sequence selected from the group of SEQ ID NO: 1, SEQ ID NO: 2, SEQ ID NO: 3, SEQ ID NO: 4, SEQ ID NO: 5, and combinations thereof.

5. The pharmaceutical composition of claim 4, wherein the recombinant polypeptide is the amino acid sequence as set forth in SEQ ID NO: 1.

6. The pharmaceutical composition of claim 1, further comprising an amino acid selected from the group consisting of cysteine, methionine, and combinations thereof.

7. The pharmaceutical composition of claim 6, further comprising a water-soluble thickening agent selected from the group consisting of carboxymethyl cellulose, dextrose, mannitol, methyl cellulose, hydroxyethyl cellulose, hydroxypropyl cellulose, non-cross-linked or partially cross-linked polyacrylates, polyoxyethylene sorbitan monolaurates, polyoxyethylene sorbitan monopalmitates, polyoxyethylene sorbitan monostearates, polyoxyethylene sorbitan monooleates, and combinations thereof.

8. The pharmaceutical composition of claim 7, further comprising at least one non-ionic poly(oxyethlene-co-oxypropylene) block copolymer.

9. The pharmaceutical composition of claim 6, further comprising at least one non-ionic poly(oxyethlene-co-oxypropylene) block copolymer.

10. The pharmaceutical composition of claim 1, further comprising a water-soluble thickening agent selected from the group consisting of carboxymethyl cellulose, dextrose, mannitol, methyl cellulose, hydroxyethyl cellulose, hydroxypropyl cellulose, non-cross-linked or partially cross-linked polyacrylates, polyoxyethylene sorbitan monolaurates, polyoxyethylene sorbitan monopalmitates, polyoxyethylene sorbitan monostearates, polyoxyethylene sorbitan monooleates, and combinations thereof.

11. The pharmaceutical composition of claim 10, further comprising at least one non-ionic poly(oxyethlene-co-oxypropylene) block copolymer.

12. The pharmaceutical composition of claim 1, further comprising at least one non-ionic poly(oxyethlene-co-oxypropylene) block copolymer.

13. The pharmaceutical composition of claim 1, wherein the composition is ensconced within substantially spherical particles fabricated from a water soluble biodegradable polymer selected from the group consisting of poly(lactic acid-co-glycolic acid), poly(lactic acid), poly(glycolic acid), poly(caprolactone), and poly(hydroxybutyrate).

14. The pharmaceutical composition of claim 13, wherein the biodegradable polymer is poly(lactic acid-co-glycolic acid).

15. A method for treating, preventing or alleviating a disease, condition, syndrome, symptom, pathology, or malady in a mammalian subject in need of such treatment comprising administering to the subject the composition of claim 1.

16. The method of claim 15, wherein the administering is accomplished by intramuscular or subcutaneous injection.

17. The method of claim 15, wherein the disease being treated is selected from the group consisting of multiple sclerosis, autoimmune diseases, and rheumatic disorders.

18. The method of claim 15, wherein the condition, syndrome, symptom, pathology, or malady comprises infantile spasms, Addison's disease, Nelson's, Cushing's and West syndromes.

19. A method for fabricating a pharmaceutical composition, comprising combining a quantity of corticotropin with a quantity of at least one water-soluble thickening agent, wherein the composition is free of gelatin and free of preservatives.

20. The method of claim 19, wherein corticotropin comprises a recombinant polypeptide and optionally a naturally occurring polypeptide.

21. The method of claim 19, wherein the corticotropin is free of a naturally occurring polypeptide.

22. The method of claim 20, wherein the recombinant polypeptide is the amino acid sequence selected from the group of SEQ ID NO: 1, SEQ ID NO: 2, SEQ ID NO: 3, SEQ ID NO: 4, SEQ ID NO: 5, and combinations thereof.

23. The method of claim 22, wherein the recombinant peptide is the amino acid sequence as set forth in SEQ ID NO: 1.
